(19) United States
(12) Patent Application Publication
(10) Pub. No.: US 2022/0387754 A1
(43) Pub. Date: Dec. 8, 2022

(54) CATHETER ASSEMBLY

(71) Applicant: TERUMO KABUSHIKI KAISHA, Tokyo (JP)

(72) Inventor: Masahiro ISHIDA, Kanagawa (JP)

(73) Assignee: TERUMO KABUSHIKI KAISHA, Tokyo (JP)

(21) Appl. No.: 17/889,323

(22) Filed: Aug. 16, 2022

Related U.S. Application Data
(63) Continuation of application No. PCT/JP2021/005954, filed on Feb. 17, 2021.

(30) Foreign Application Priority Data
Feb. 20, 2020 (JP) 2020-026893

(51) Int. Cl. A61M 25/00; A61M 25/06

(52) U.S. Cl. CPC A61M 25/0097; A61M 25/0014; A61M 25/0606

(57) ABSTRACT

A catheter assembly includes a catheter, a catheter hub fixed to the catheter, an inner needle inserted through the catheter, and a grip that fixes and holds the inner needle. In addition, the catheter assembly includes a support structure portion that supports the outer side of the catheter. The support structure portion comes into contact with the catheter in an assembled state to deform at least one of the support structure portion and the catheter.

### CROSS-REFERENCE TO RELATED APPLICATIONS

This is a bypass continuation of PCT Application No. PCT/JP2021/005954, filed on Feb. 17, 2021, which claims priority to Japanese Patent Application No. JP2020-026893, filed on Feb. 20, 2020. The contents of these application are hereby incorporated by reference in their entireties.

### BACKGROUND

The present disclosure relates to a catheter assembly having a structure capable of supporting the outer side of a catheter at the time of puncture with the catheter and an inner needle.

When an introduction unit for infusion, blood transfusion, or the like is constructed in a treatment target (patient), a catheter assembly as disclosed in US 2016/0256667 A is used. The catheter assembly includes a multi-structure needle in which an inner needle is inserted into a catheter (outer needle). When the catheter assembly is used, a user punctures the multi-structure needle into a body of the patient, then causes the catheter to enter a blood vessel, and further removes the inner needle from the catheter to indwell the catheter.

During use, this type of catheter assembly is likely to deflect when a user brings a portion of the multi-structure needle exposed from a grip into contact with the treatment target to obliquely puncture the treatment target with the multi-structure needle. When the multi-structure needle is deflected in this manner, it becomes difficult to puncture the multi-structure needle. Therefore, the catheter assembly disclosed in US 2016/0256667 A has a structure for supporting the catheter by arranging a distal end of the grip to which the inner needle is fixed at a position near the outer periphery of the catheter.

### SUMMARY

The catheter in the assembly described above is typically manufactured by extrusion molding or the like, and thus, a difference in thickness is likely to occur even within a range of a dimensional tolerance. Therefore, a support portion that supports the catheter is formed to have a space larger than the maximum tolerance of an outer diameter of the catheter such that a clearance is generated between the support portion and an outer peripheral surface of the catheter. However, if the catheter is manufactured to be thin, the clearance between the outer peripheral surface of the catheter and the support portion of the grip becomes large, and there is a disadvantage that the support portion is not capable of effectively supporting the catheter.

The present disclosure relates to the above-described catheter assembly technique, and an object thereof is to provide a catheter assembly capable of absorbing a tolerance accompanying manufacture of a catheter and favorably supporting the outer side of the catheter.

According to one aspect of the disclosure, a catheter assembly includes: a catheter; a catheter hub fixed to the catheter; an inner needle inserted through the catheter; a grip that fixes and holds the inner needle; and a support structure portion that holds the outer side of the catheter in an assembled state. In the assembled state, the support structure portion sandwiches the catheter, and at least one of the support structure portion and the catheter is elastically deformed.

The above-described catheter assembly can favorably support the outer side of the catheter by absorbing the tolerance accompanying the manufacture of the catheter.

### BRIEF DESCRIPTION OF DRAWINGS

FIG. 1 is a perspective view of a catheter assembly according to one embodiment of the present invention;

FIG. 2 is an exploded perspective view of the catheter assembly of FIG. 1;

FIG. 3 is a perspective view of a catheter operation member as viewed from obliquely below;

FIG. 4 is a partial front view illustrating a distal portion of the catheter assembly;

FIG. 5 is a side view illustrating the distal portion of the catheter assembly;

FIG. 6A is a front sectional view illustrating an assembled state of a catheter and a support structure portion; FIG. 6B is a front sectional view illustrating an assembled state of a catheter and a support structure portion according to a first modification; FIG. 6C is a front sectional view illustrating an assembled state of a catheter and a support structure portion according to a second modification;

FIG. 7A is a front sectional view illustrating a state before assembly and in an assembled state of a catheter and a support structure portion according to a third modification; FIG. 7B is a front sectional view illustrating a state before assembly and an assembled state of a catheter and a support structure portion according to a fourth modification;

FIG. 8A is a side view illustrating a state of the catheter assembly at the time of puncture; FIG. 8B is a side view illustrating a state in which the catheter operation member is advanced after puncture;

FIG. 9 is a partial perspective view schematically illustrating a distal end of a catheter assembly having a support structure portion according to a fifth modification of the present invention;

FIG. 10A is a front sectional view illustrating a state before assembly and in an assembled state of a catheter and the support structure portion according to the fifth modification; and FIG. 10B is a front sectional view illustrating a state before assembly and an assembled state of a catheter and a support structure portion according to a sixth modification.

### DETAILED DESCRIPTION

Hereinafter, preferred embodiments of the present invention will be described in detail with reference to the accompanying drawings.

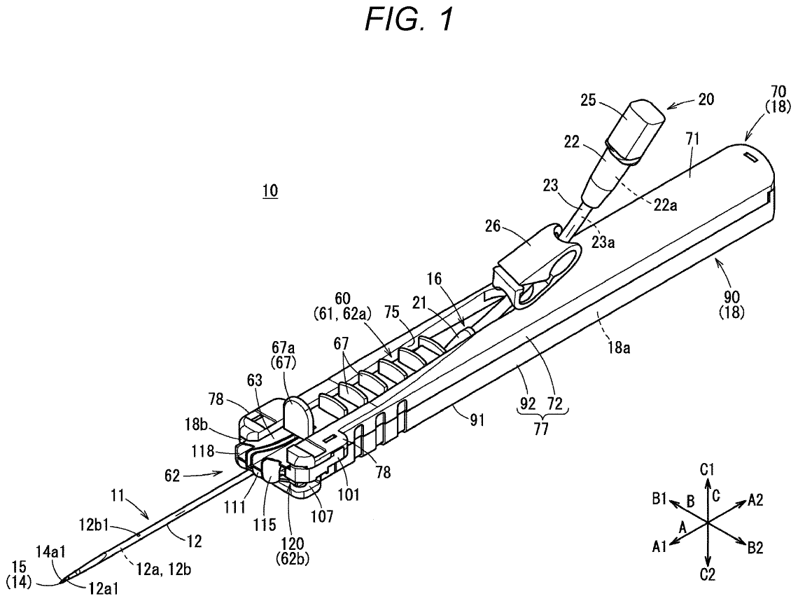

As illustrated in FIG. 1, a catheter assembly 10 according to an embodiment of the present invention is a medical device used at the time of performing infusion, blood transfusion, blood sampling, or the like on a treatment target (living body), and a catheter 12 is inserted into and indwelled inside the body of the treatment target to cause the inside and outside of the body to communicate. This catheter assembly 10 enables insertion of a catheter 12 having a longer length than a peripheral venous catheter (for example, a central venous catheter, a PICC, a mid-line catheter, and the like). Incidentally, the catheter assembly 10 may also be configured to enable insertion of the peripheral venous catheter. In addition, the catheter assembly 10 is not limited to the venous catheter, and may be configured to enable insertion of an arterial catheter such as a peripheral arterial catheter.

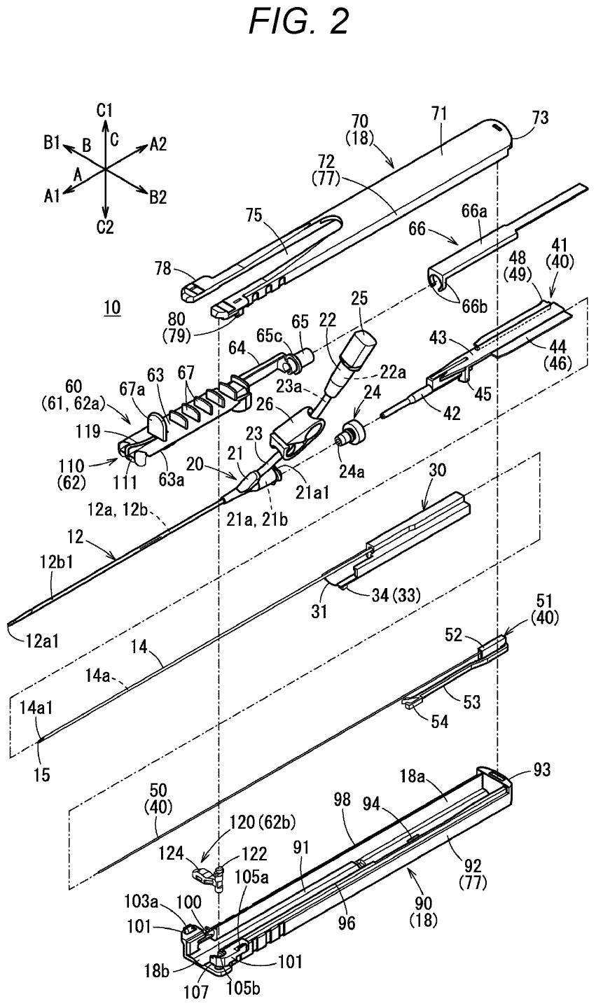

As illustrated in FIGS. 1 and 2, the catheter assembly 10 includes an inner and outer needle assembly 16 in which the catheter 12, an inner needle 14, a catheter hub 20, an inner needle hub 30, a safety member 40, and a catheter operation member 60 are assembled in a state before use (before puncture). Further, the catheter assembly 10 includes a grip 18 (housing) configured to accommodate the inner and outer needle assembly 16 and be gripped by a user.

In the inner and outer needle assembly 16, in the state before puncture, the inner needle 14 penetrates through the catheter 12 and the catheter hub 20 to form a multi-structure needle 11, and a needle tip 15 of the inner needle 14 protrudes from a distal end of the catheter 12. The safety member 40 through which the inner needle 14 is inserted is arranged closer to a proximal side than the catheter hub 20, and the inner needle hub 30 holding the inner needle 14 is arranged on the proximal side of the safety member 40. The catheter operation member 60 is arranged above the catheter 12, the catheter hub 20, and the safety member 40, and causes advancement and retraction of these members under the operation of the user. The inner and outer needle assembly 16 including a portion of the multi-structure needle 11 on the proximal side is accommodated in the grip 18, and the inner needle hub 30 is fixed to the grip 18.

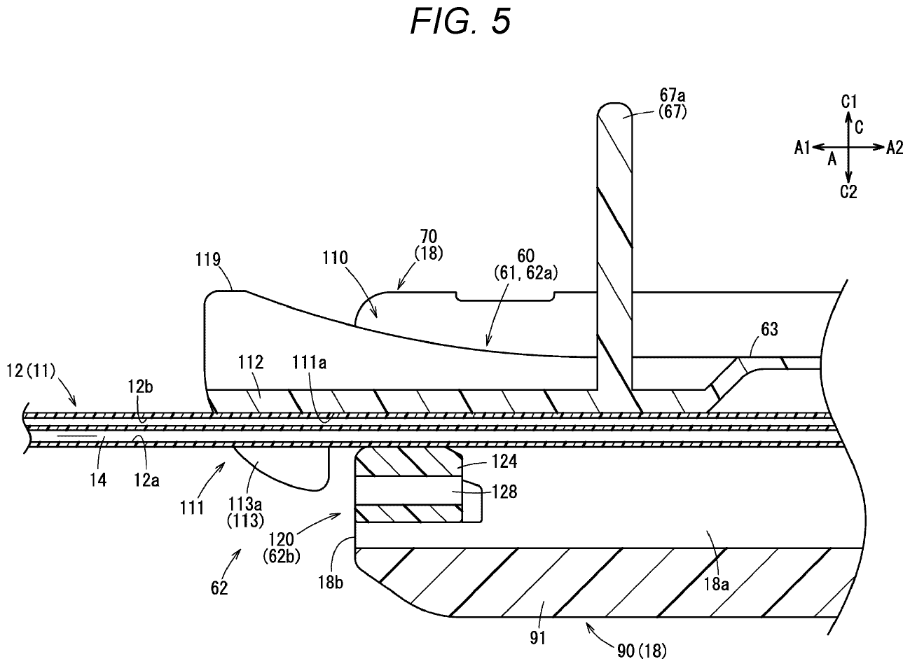

The catheter 12 according to the present embodiment is a tubular body having appropriate flexibility, and is configured as a multi-lumen type having a plurality of lumens 12a and 12b therein (see also FIG. 5). The lumens 12a and 12b extend in the axial direction (arrow A direction) of the catheter 12, and communicate with a distal opening 12a1 and a distal opening 12b1, respectively, at the distal end of the catheter 12. For example, the lumen 12a is formed in a circular shape capable of accommodating the inner needle 14, and the lumen 12b is formed in an arc-shaped ellipse above the lumen 12a. A length of the catheter 12 is set to about 14 to 500 mm, preferably within a range of 30 to 400 mm, and more preferably within a range of 76 to 200 mm.

A constituent material of the catheter 12 is a soft resin material is suitable, and examples thereof include a fluorine-based resin such as polytetrafluoroethylene (PTFE), ethylene-tetrafluoroethylene copolymer (ETFE) and perfluoroalkoxy fluorine resin (PFA), an olefin-based resin such as polyethylene and polypropylene or a mixture thereof, polyurethane, polyester, polyamide, polyether nylon resin, a mixture of the olefin-based resin and ethylene-vinyl acetate copolymer, and the like. Note that it is a matter of course that the catheter 12 may be a single lumen type including only the lumen 12a through which the inner needle 14 is inserted without being limited to the multi-lumen type.

A proximal end of the catheter 12 is fixed to a distal end in the catheter hub 20 by an appropriate fixing means such as caulking, fusion, and adhesion. The catheter hub 20 is exposed on a skin of a treatment target in a state in which the catheter 12 has been inserted into a blood vessel of the treatment target, and indwells together with the catheter 12 by being pasted with a tape or the like.

The catheter hub 20 has two separate hubs (main hub 21 and sub hub 22) corresponding to the multi-lumen type catheter 12. The main hub 21 is a member directly connected to the catheter 12, and the sub hub 22 is a member connected to the main hub 21 through a tube 23.

A constituent material of the catheter hub 20 (main hub 21 and sub hub 22) is not particularly limited, but a thermoplastic resin such as polypropylene, polycarbonate, polyamide, polysulfone, polyarylate, and a methacrylate-butylene-styrene copolymer may be preferably applied, for example.

The main hub 21 is a tubular body extending in parallel with the axis on the proximal side of the catheter 12, and the tube 23 is connected to a predetermined position on an outer peripheral surface of the main hub 21. An internal space 21a communicating with the lumen 12a and an internal space 21b communicating with the lumen 12b are provided inside the main hub 21. A proximal end of the internal space 21a communicates with a proximal opening 21a1 of the main hub 21. On the other hand, the internal space 21b is separated from the internal space 21a and communicates with a lumen 23a of the tube 23 inserted into and fixed to the main hub 21.

In the state before puncture, a valve member 24 is inserted into the main hub 21 from the proximal opening 21a1 toward the back side (arrow A1 side) of the internal space 21a. A valve hole 24a that can be elastically opened and closed is provided at the axial center of the valve member 24. Distal ends of the inner needle 14 and the safety member 40 are inserted into the valve hole 24a in the state before puncture of the valve member 24, so that an inner surface of the valve hole 24a and an outer surface of the safety member 40 are in close contact with each other. As a result, the valve member 24 brings the catheter hub 20 and the safety member 40 into a fitted state, and prevents leakage of blood from the proximal opening 21a1 of the main hub 21 at the time of puncture with the inner needle 14.

In addition, the sub hub 22 is formed in a tubular shape having the same thickness as the main hub 21, and a proximal end of the tube 23 is inserted from a distal end of the sub hub 22 and fixed thereto. An internal space 22a communicating with the lumen 23a of the tube 23 is formed inside the sub hub 22. A proximal end of the internal space 22a communicates with a proximal opening (not illustrated) of the sub hub 22. In the state before puncture, a closing member 25 that closes the proximal opening is connected to the sub hub 22.

The tube 23 is configured to have flexibility similarly to the catheter 12. A clamp 26 capable of opening and closing the lumen 23a of the tube 23 is attached in advance to an intermediate position in the extending direction of the tube 23.

Meanwhile, the inner needle 14 of the catheter assembly 10 is configured as a hollow tubular body having rigidity capable of puncturing a skin of a living body. The needle tip 15, which is sharp, is formed at the distal end of the inner needle 14. A hollow portion 14a is formed inside the inner needle 14 to penetrate along the arrow A direction, and the hollow portion 14a communicates with a distal opening 14a1 provided in the needle tip 15.

Examples of a constituent material of the inner needle 14 include a metal material such as stainless steel, aluminum or an aluminum alloy, and titanium or a titanium alloy, a hard resin, ceramics, and the like. The inner needle 14 is firmly fixed to the inner needle hub 30 by an appropriate fixing means such as fusion, adhesion, and insert molding.

The inner needle hub 30 directly holds the inner needle 14 and is fixed to the grip 18 through a grip fixing portion 31 (lower wall) formed on an arrow C2 side. The grip fixing portion 31 has a lower surface provided with a plurality of fixing protrusions 34 that protrude shortly in the downward direction and constitute an attachment mechanism 33 with the grip 18.

The safety member 40 is configured to be inserted and fitted into the main hub 21 and the valve member 24 of the catheter hub 20 to follow the catheter hub 20 that is moving. The safety member 40 includes a cover body 41 that covers the outer side of the inner needle 14 along with advancement, a blunt needle 50 protruding from the needle tip 15 of the inner needle 14 after puncture, and the blunt needle hub 51 holding the blunt needle 50.

The cover body 41 includes: a distal cover portion 42 that has a cylindrical shape and accommodates and protects the inner needle 14 after puncture; a proximal extending portion 43 extending from an upper portion of the distal cover portion 42 to the arrow A2 side; and a pair of protruding pieces 44 protruding outward in the width direction from the proximal extending portion 43. In addition, an engagement projection 45 with which the blunt needle hub 51 is engaged is provided at a point where the proximal extending portion 43 and the distal cover portion 42 are connected.

The distal cover portion 42 is inserted into and brought into close contact with the valve member 24 on the distal side, thereby being frictionally fitted to the catheter hub 20 including the valve member 24. In the state before puncture, a proximal end of the distal cover portion 42 faces a distal end of the inner needle hub 30. The proximal extending portion 43 connected to the distal cover portion 42 extends to a proximal end in the grip 18 along an upper portion of the inner needle hub 30 in the state before puncture.

The pair of protruding pieces 44 protrudes outward in the width direction (arrow B direction) from the inner needle hub 30 and extends to the vicinity of a side wall 77 of the grip 18 (above rail walls 96 and 98 to be described later). The respective protruding pieces 44 constitute a guide mechanism 46 that performs guidance in the arrow A direction in cooperation with the grip 18 when the safety member 40 moves. In addition, a locked protrusion 48 is provided on a side of the protruding piece 44 on an arrow B1 side. The locked protrusion 48 is locked to a locking portion 100 of the grip 18 at the advanced position at which the safety member 40 has advanced, thereby constituting one of safety movement restricting mechanisms 49 that restrict the advancement and retraction of the cover body 41.

The blunt needle 50 of the safety member 40 is a rod member (round rod) configured to prevent erroneous puncture of the catheter 12 and the inner needle 14 with respect to a living body, and is movably accommodated in the hollow portion 14a of the inner needle 14. A distal end of the blunt needle 50 is formed in a shape blunter than the needle tip 15 of the inner needle 14 (for example, to have a polished flat surface), and is arranged at a position near a proximal end of the distal opening 14a1 in the hollow portion 14a of the inner needle 14 in the state before puncture. The distal end of the blunt needle 50 is exposed from the needle tip 15 (distal opening 14a1) as the safety member 40 advances.

The blunt needle hub 51 is configured to be relatively movable with respect to the inner needle 14, the inner needle hub 30, and the grip 18 by holding the blunt needle 50 and engaging with the engagement projection 45 of the cover body 41. The blunt needle hub 51 includes a blunt needle holding portion 52 that holds the blunt needle 50 on the arrow A2 side, and an arm portion 53 extending from the blunt needle holding portion 52 to the arrow A1 side.

The blunt needle holding portion 52 is arranged in a space on the proximal side of a point where the inner needle 14 is fixed in the inner needle hub 30. When a distal end surface of the blunt needle holding portion 52 comes into contact with the fixing point of the inner needle 14 as the blunt needle hub 51 advances, the subsequent advancement of the blunt needle hub 51 is prevented.

The entire extending portion of the arm portion 53 is configured to be elastically deformable in the width direction, and an engagement end 54 to be engaged with the engagement projection 45 in the state before puncture is provided at the distal end of the arm portion 53. When the cover body 41 further advances at a stage where the movement of the blunt needle hub 51 is restricted, the engagement end 54 is elastically deformed as appropriate to release the engagement with the engagement projection 45.

Note that the safety member 40 is not limited to the above configuration as long as erroneous puncture of the needle tip 15 of the inner needle 14 can be prevented. For example, the safety member 40 may include only the cover body 41 without including the blunt needle 50 and the blunt needle hub 51.

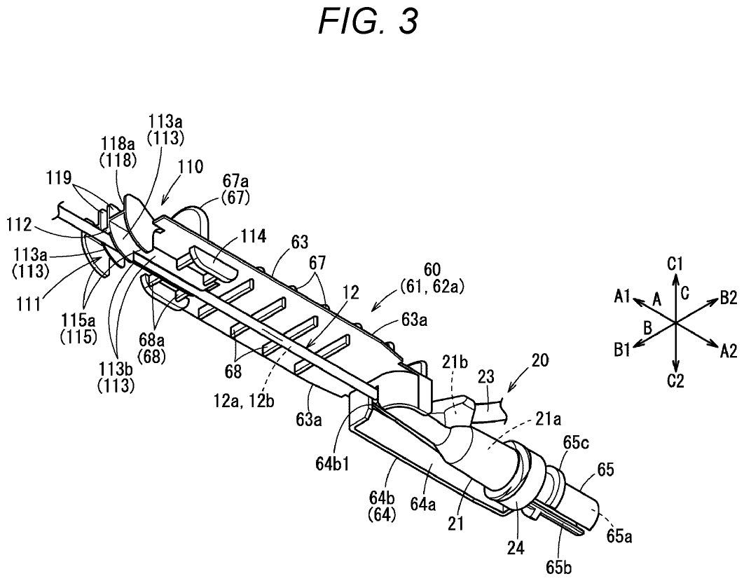

As illustrated in FIGS. 2 and 3, the catheter operation member 60 constitutes an operation unit 61 to be operated by the user in the catheter assembly 10. In addition, the catheter operation member 60 according to the present embodiment is also a first support portion 62a of a support structure portion 62 that supports the outer side of the catheter 12 (multi-structure needle 11) at the time of puncture with the multi-structure needle 11. A material forming the catheter operation member 60 is not particularly limited, and, for example, the materials exemplified for the catheter hub 20 can be appropriately selected.

Specifically, the catheter operation member 60 includes: an operation plate portion 63 (extending portion) extending in the arrow A direction; a hub engagement portion 64 connected to a proximal end of the operation plate portion 63 and engaged with the catheter hub 20; and an operation member tubular portion 65 that is connected to a proximal end of the hub engagement portion 64 and accommodates the safety member 40. In addition, the catheter operation member 60 includes a cover member 66 that extends in the proximal direction from the hub engagement portion 64 and covers the safety member 40.

The operation plate portion 63 is a site in contact with a user's finger to perform an advancing/retracting operation. The operation plate portion 63 is formed to be thin and has flexibility capable of being curved in a direction away from the multi-structure needle 11. The operation plate portion 63 has side edges 63a, which extend in the arrow A direction, on both sides in the width direction. A plurality of tabs 67 are provided on an upper surface of the operation plate portion 63. A tab 67a at the most distal end among the plurality of tabs 67 protrudes more than the other tabs 67. Further, a plurality of ribs 68 protrude shortly from a lower surface of the operation plate portion 63. The catheter 12 is arranged below the plurality of ribs 68.

An operation support portion 110 configured to support the catheter 12 is provided in a distal region of the operation plate portion 63. The operation support portion 110 is formed on the lower surface side of the operation plate portion 63, and has an upper support portion 111 that arranges the catheter 12 (multi-structure needle 11) inward in the width direction.

The upper support portion 111 is provided at the central portion in the width direction of the catheter operation member 60 and extends from a distal end toward the arrow A2 side by a predetermined length. A proximal end of the upper support portion 111 reaches the arrow A2 side with respect to the tab 67a. The upper support portion 111 includes: a base portion 112 that can come into contact with an arrow C1 side of the catheter 12; and a pair of ridge portions 113 that can come into contact with the catheter 12 in the arrow B direction.

The base portion 112 is formed to be slightly shifted to the lower side (arrow C2 side) from the operation plate portion 63, and a vertical position of a lower surface facing the catheter 12 substantially coincides with a protruding end of the rib 68.

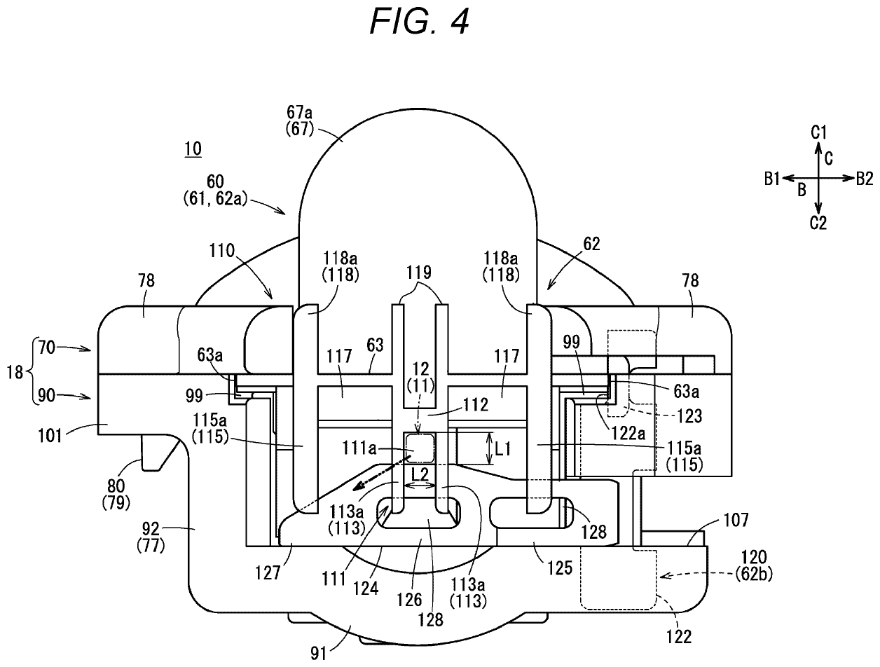

The pair of ridge portions 113 is configured to sandwich the base portion 112 therebetween, and protrudes from the lower surface of the operation plate portion 63 to the arrow C2 side. A protruding amount of each of the ridge portions 113 from the operation plate portion 63 is larger than a protruding amount of the base portion 112 from the operation plate portion 63 (see also FIG. 4).

In addition, each of the ridge portions 113 is formed in a stepped shape in which a portion on the distal side protrudes toward the arrow C2 side from a portion on the proximal side. The portion on the distal side (hereinafter, referred to as a distal projecting portion 113a) of the step is formed to be sufficiently longer toward the arrow C2 side than an arrangement position of the catheter 12. An edge portion of each of the distal projecting portions 113a is curved toward the proximal side (arrow A2 side) from a distal end connected to the operation plate portion 63 to the arrow C2 side, and linearly extends to the arrow C1 side at a protruding top on the arrow C2 side.

A lower edge portion of the step on the portion on the proximal side (hereinafter, referred to as a proximal projecting portion 113b) is continuous with the edge portion of the distal projecting portion 113a and linearly extends toward the arrow A2 side. Each of the proximal projecting portions 113b protrudes from the base portion 112 by a length substantially equal to an outer diameter of the catheter 12. Some ribs 68a on the distal side among the plurality of ribs 68 provided on the lower surface of the operation plate portion 63 are provided to be continuous with the respective proximal projecting portions 113b. These ribs 68a are continuous with short side ribs 114 extending in the arrow A direction on the outer side in the width direction. The side rib 114 protrudes to the arrow C2 side more than the rib 68a, and has a function of guiding the advancement and retraction of the catheter operation member 60 with respect to the grip 18.

In the upper support portion 111 configured as described above, the catheter 12 is arranged in a support space 111a formed by the base portion 112 and the pair of ridge portions 113 in the state before puncture (assembled state). That is, the catheter 12 is covered by the base portion 112 on the upper side and covered by the pair of ridge portions 113 on the right and left sides in the width direction. Since the upper support portion 111 is long in the arrow A direction, the catheter 12 is favorably maintained in a linearly extending state in a long range of a distal region of the catheter operation member 60.

In addition, the operation support portion 110 includes a pair of lower protruding blocks 115 protruding toward the arrow C2 side on the lower surface side of the operation plate portion 63 and on the outer side of the upper support portion 111 in the width direction. Each of the lower protruding blocks 115 is provided with a predetermined gap 117 with respect to the upper support portion 111.

The pair of lower protruding blocks 115 also protrudes toward the arrow C2 side to the same extent as the distal projecting portions 113a of the pair of ridge portions 113. In addition, a width (thickness) of each of the lower protruding blocks 115 is larger than a width (thickness) of the ridge portion 113. A distal end surface 115a of each of the lower protruding blocks 115 is formed to be curved to the arrow A2 side as proceeding to the arrow C2 side.

That is, each of the distal projecting portions 113a and each of the lower protruding blocks 115 are at positions overlapping each other in a side view, and are formed in substantially the same shape. As a result, the lower distal side (the arrow A1 side and the arrow C2 side) of the catheter operation member 60 are guided such that the catheter operation member 60 is separated from the catheter 12 when the catheter operation member 60 is delivered from the grip 18 to some extent and comes into contact with a body surface of the treatment target.

Further, the operation support portion 110 has a pair of upper protruding blocks 118, which protrude shortly toward the arrow C1 side at the same position as the lower protruding block 115 in the width direction on the upper surface side of the operation plate portion 63. A width of each of the upper protruding blocks 118 coincides with the width of each of the lower protruding blocks 115. A distal end surface 118a of each of the upper protruding blocks 118 is flush with an upper end of the distal end surface 115a of the lower protruding block 115.

Further, a pair of reinforcing pieces 119 (reinforcing portions) is provided on the inner side of the pair of upper protruding blocks 118 in the width direction. Each of the reinforcing pieces 119 is provided at the same position as each of the ridge portions 113 in the width direction and protrudes to the opposite side (arrow C1 side) of each of the ridge portions 113. Each of the reinforcing pieces 119 protrudes from the upper surface of the operation plate portion 63 to the same extent as the upper protruding block 118 at the distal end, is formed to gradually decline while being curved from the distal end to the arrow A2 side, and is in contact with the tab 67a. Since the operation plate portion 63 does not exist between the respective reinforcing pieces 119, a groove portion in which an upper surface of the base portion 112 of the upper support portion 111 is exposed is formed.

Meanwhile, the hub engagement portion 64 continuous with the proximal end of the operation plate portion 63 has an accommodation chamber 64a for accommodating the main hub 21, and has a wall portion 64b on the arrow B1 side to be formed in a shape in which the arrow B2 side is cut out (shape in which the accommodation chamber 64a is opened). This shape is formed to expose the sub hub 22 and the tube 23 of the catheter hub 20 configured as the multi-lumen type. The distal side of the hub engagement portion 64 extends such that the wall portion 64b on the arrow B1 side wraps around the accommodation chamber 64a. The wall portion 64b on the distal side is provided with a gap 64b1 that is narrower than a diameter of the catheter hub 20 and from which only the catheter 12 (multi-structure needle 11) extends.

The operation member tubular portion 65 is formed in a cylindrical shape that protrudes shortlyin the proximal direction from a proximal end surface of the hub engagement portion 64. A communicating space 65a that communicates with the accommodation chamber 64a and in which the safety member 40 (cover body 41) is arranged is provided on the inner side of the operation member tubular portion 65. In addition, a slit 65b communicating with the accommodation chamber 64a and the communicating space 65a is formed in the lower portion of the operation member tubular portion 65. Further, an arcuate rib 65c that protrudes in the circumferential direction and restricts the movement of the cover member 66 is provided on the outer peripheral surface of the operation member tubular portion 65.

Returning to FIG. 2, the cover member 66 is mounted on the catheter operation member 60 to prevent the user from directly coming into contact with the safety member 40. The cover member 66 includes a body portion 66a that covers the upper side (side on which a hand is located when being gripped by the user) of the safety member 40, and a pair of attachment leg portions 66b provided at a distal end of the body portion 66a and attached to the operation member tubular portion 65. The pair of attachment leg portions 66b is engaged between the proximal end surface of the hub engagement portion 64 and the arcuate rib 65c.

In addition, the grip 18 of the catheter assembly 10 is formed to have an appropriate thickness that can be easily held by the user, and extends along the arrow A direction. The accommodation space 18a in which the catheter 12, the catheter hub 20, the safety member 40, and the catheter operation member 60 can advance and retract is formed in the grip 18. The accommodation space 18a communicates with a distal opening portion 18b of the grip 18. The grip 18 is configured by assembling an upper grip 70 and a lower grip 90, which can be divided in the arrow C direction, with each other.

The upper grip 70 has an upper wall 71, a pair of upper side walls 72, and an upper rear wall 73, and is formed in a recessed shape (bowl shape) that is opened downward. The pair of upper side walls 72 constitutes side walls 77 on both sides in the width direction of the grip 18 together with a lower side wall 92 of the lower grip 90.

In addition, the upper wall 71 has an operation member exposure notch 75 at the center in the arrow B direction closer to the distal side than an intermediate portion in the arrow A direction. The operation member exposure notch 75 is opened at the distal end to communicate with the accommodation space 18a, and exposes the tube 23 of the catheter hub 20 and the tab 67 of the catheter operation member 60 so as to be capable of advancing and retracting. Further, the upper grip 70 has a pair of upper projecting pieces 78 at the distal end, and a fixing hook 80 constituting a fixing mechanism 79 between the upper grip 70 and the lower grip 90 is provided on lower surfaces of the respective upper projecting pieces 78 and the upper rear wall 73.

The lower grip 90 has a bottom wall 91, a pair of lower side walls 92, and a lower rear wall 93, and is formed in a recessed shape (bowl shape) opened upward. A predetermined range on the arrow A2 side of the bottom wall 91 is a mounting target portion on which the inner needle hub 30 is mounted, and a plurality of (three in the present embodiment) mounting holes 94 (a part of the attachment mechanism 33) into which the fixing protrusion 34 of the inner needle hub 30 can be fitted are provided.

In addition, the pair of lower side walls 92 has the rail walls 96 and 98 in the upper portion thereof, and the upper side wall 72 of the upper grip 70 is arranged on the outer side of the rail walls 96 and 98 in the width direction in an assembled state. In the assembled state, the pair of side edges 63a of the catheter operation member 60 and the protruding pieces 44 of the safety member 40 are slidably arranged on the pair of rail walls 96 and 98. In addition, the locked protrusion 48 of the cover body 41 is arranged between the upper side wall 72 on the arrow B1 side and the rail wall 98.

The lower side wall 92 on the arrow B1 side is provided with a locking portion 100 that is engaged with the locked protrusion 48 to define an advancement limit of the cover body 41 and restrict the retraction of the cover body 41 when the cover body 41 of the safety member 40 advances. That is, the locking portion 100 constitutes the safety movement restricting mechanism 49 together with the locked protrusion 48. The catheter assembly 10 can favorably cover (protect) the inner needle 14 after puncture with the cover body 41 by restricting detachment of the safety member 40 from the grip 18.

A pair of projecting bodies 101 protruding outward in the width direction from the pair of lower side walls 92 is provided at the distal end of the lower grip 90. The projecting body 101 on the arrow B1 side has a first fixing hole 103a that is a part of the fixing mechanism 79. The projecting body 101 on the arrow B2 side has a second fixing hole 105a that is a part of the fixing mechanism 79. In addition, the projecting body 101 on the arrow B2 side has a movement space 107 in which the lower support member 120 is rotatable.

As illustrated in FIGS. 2, 4, and 5, the lower support member 120 is rotatably attached to the grip 18, and constitutes a second support portion 62b of the support structure portion 62 that supports the catheter 12 (multi-structure needle 11) extending below the catheter operation member 60 from below in the state before puncture. In addition, the lower support member 120 rotates by contact with the wall portion 64b of the hub engagement portion 64 when the catheter operation member 60 advances, and enables the catheter operation member 60 (and the catheter hub 20 and the safety member 40) to be delivered from the accommodation space 18a.

The lower support member 120 includes a shaft 122 extending in the arrow C direction and a lower support portion 124 protruding in a direction orthogonal to the axial center of the shaft 122. The shaft 122 has an upper portion provided with a guide plane 122a continuous with an upper end of the rail wall 96 and a pair of small projections 123 having the guide plane 122a. The side edge 63a of the catheter operation member 60 is close to the guide plane 122a in the state before puncture. As a result, the rotation of the lower support member 120 is restricted.

The lower support portion 124 has an appropriate thickness in the arrow C direction, and is connected to the lower side (arrow C2 side) of an intermediate position of the shaft 122 in the arrow C direction. The lower support portion 124 is formed to be thicker than a thickness of the bottom wall 91 of the lower grip 90, for example. The lower support portion 124 is formed by providing a coupling portion 125 connected to the shaft 122, a central portion 126 located at the central portion in the width direction, and a protruding end 127 located at a position farthest from the shaft 122 to be continuous with each other from the arrow B2 side to the arrow B1 side.

The coupling portion 125 and the central portion 126 are provided respectively with lightening holes 128 penetrating in the arrow A direction in order to enhance the molding accuracy of the lower support portion 124. The central portion 126 slightly protrudes to the arrow C1 side from the coupling portion 125 and the protruding end 127, and has the largest thickness in the lower support portion 124. The lower support portion 124 basically supports the lower side of the catheter 12 at the central portion 126. An upper surface of the protruding end 127 is gradually inclined downward from the central portion 126 to the arrow B1 side.

The lower support member 120 configured as described above is inserted along the bearing notch 105b from above the lower grip 90 in a posture in which the lower support portion 124 faces the arrow B1 side. At this time, the lower support member 120 is smoothly inserted into the bearing notch 105b as the vicinity of the coupling portion of the lower support portion 124 passes through an opening portion of the bearing notch 105b on the accommodation space 18a side. When the upper grip 70 and the lower grip 90 are mounted, the upper end of the shaft 122 supported by the lower grip 90 is pivotally supported by the upper grip 70.

In the state before puncture of the lower support member 120, the side edge 63a of the catheter operation member 60 is present on the guide plane 122a, and thus, the lower support portion 124 is restricted from rotating and stands by to be capable of supporting the catheter 12. As a result, the lower support portion 124 supports the catheter 12 from below to suppress the deflection of the catheter 12. When the catheter operation member 60 advances from the grip 18, the lower support member 120 becomes rotatable as the side edge 63a comes out of the guide plane 122a, and allows the catheter hub 20, the catheter operation member 60, and the safety member 40 to be delivered.

In the catheter assembly 10 configured as described above, the inner and outer needle assembly 16 (catheter 12, inner needle 14, catheter hub 20, inner needle hub 30, safety member 40, and catheter operation member 60) is assembled first at the time of assembly. Thereafter, the inner and outer needle assembly 16 is arranged on the lower grip 90 incorporating the lower support member 120, and the upper grip 70 is mounted on the inner and outer needle assembly 16 and the lower grip 90.

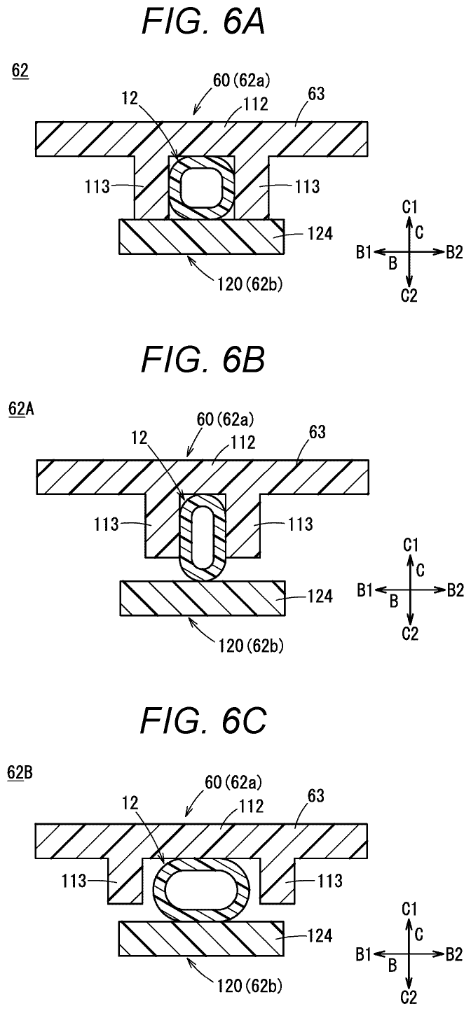

As illustrated in FIGS. 4 and 6A, the support structure portion 62 on the distal side of the grip 18 comes into contact with the outer peripheral surface of the catheter 12 and elastically deforms and holds the catheter 12 in the assembled state of the catheter assembly 10.

Note that FIG. 6A (and subsequent FIGS. 6B to 7B, 10A, and 10B) is a front sectional view schematically illustrating the catheter 12 and the support structure portion 62 (including the catheter operation member 60 or the grip 18), and illustrates the catheter 12 as a single lumen type to facilitate understanding of the invention.

The support space 111a of the support structure portion 62 is surrounded by the base portion 112, the pair of ridge portions 113, and the lower support portion 124 to be formed in a rectangular shape (substantially a square shape) in a front sectional view. In addition, the catheter operation member 60 in the assembled state is prevented from moving to the arrow C1 side by the upper side wall 72 of the upper grip 70 as the side edge 63a is located in a guide space 99 of the grip 18. Therefore, the rectangular shape of the support space 111a is stably maintained.

The support structure portion 62 (the base portion 112, the pair of ridge portions 113, and the lower support portion 124) has higher rigidity than the catheter 12, and a cross-sectional area of the support space 111a is set to be smaller than a cross-sectional area of an outer shape of the catheter 12. That is, a longitudinal interval L1 between the base portion 112 and the lower support portion 124 is smaller than the outer diameter of the catheter 12, and a lateral interval L2 between the pair of ridge portions 113 is smaller than the outer diameter of the catheter 12.

Therefore, the outer shape of the catheter 12 arranged in the support structure portion 62 in the assembled state is deformed into a substantially square shape with rounded corners in accordance with the support space 111a (see a two-dot chain line in FIG. 4). That is, the outer shape of the catheter 12 has a perfect circular cross-sectional shape before assembly (in the state of not being supported by the support structure portion 62), but is compressed in the vertical direction and the width direction by the support structure portion 62 due to the assembly and elastically deformed so as to have a flat portion. As a result, the support structure portion 62 firmly holds the catheter 12 (with no clearance between the catheter 12 and the support structure portion 62).

In addition, the distal projecting portions 113a of the pair of ridge portions 113 are arranged on the distal side of the grip 18 and the lower support member 120 in the catheter assembly 10. In addition, the distal projecting portion 113a protrudes greatly downward (to the arrow C2 side) from the support space 111a (the upper surface of the central portion 126 of the lower support portion 124) in a front view. As a result, for example, the catheter 12 can be prevented from moving obliquely downward (see the dotted arrow in FIG. 4) to come out of the support space 111a.

Note that the catheter 12 may be deformed into another shape without being deformed into the rectangular shape by the support structure portion 62 in the catheter assembly 10. For example, as in a support structure portion 62A of a first modification illustrated in FIG. 6B, the pair of ridge portions 113 may sandwich and deform the catheter 12 in the width direction (arrow B direction) without deforming the catheter 12 by the base portion 112 and the lower support portion 124. As a result, the catheter 12 is elastically deformed into a vertically long elliptical shape having a flat portion in the arrow C direction. Conversely, as in a support structure portion 62B of a second modification illustrated in FIG. 6C, the base portion 112 and the lower support portion 124 may sandwich and deform the catheter 12 in the longitudinal direction (arrow C direction) without deforming the catheter 12 by the pair of ridge portions 113. As a result, the catheter 12 is deformed into a horizontally long elliptical shape having a flat portion in the arrow B direction.

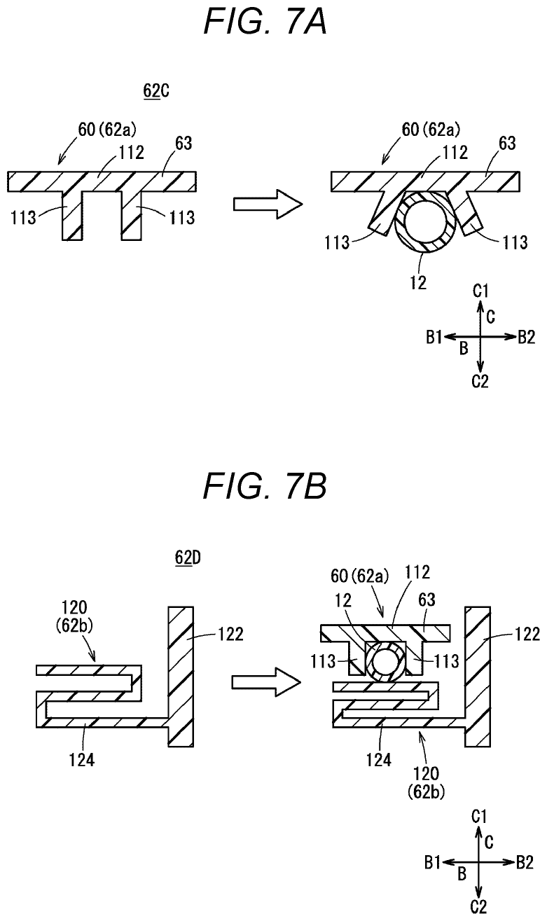

In addition, as illustrated in FIGS. 7A and 7B, the catheter assembly 10 may be configured such that not only the catheter 12 is deformed but also a support structure portion 62C or 62D itself is deformed by the catheter 12.

For example, the support structure portion 62C of a third modification illustrated in FIG. 7A is configured such that a width between the pair of ridge portions 113 of the catheter operation member 60 is smaller than the sum (the outer diameter of the catheter 12) of two wall thicknesses of the catheter 12 and the diameter of the inner needle 14. In the support structure portion 62C, the catheter 12 is arranged in the support space 111a and sandwiched between the pair of ridge portions 113 at the time of assembly, so that the pair of ridge portions 113 is elastically deformed so as to be separated from each other (in directions to be aligned with each other). In this manner, even if the support structure portion 62C side is deformed by the catheter 12, the support structure portion 62C can firmly hold the catheter 12 (with no clearance between the catheter 12 and the support structure portion 62C).

In addition, for example, the support structure portion 62D of a fourth modification illustrated in FIG. 7B is configured such that an interval between the lower support portion 124 of the lower support member 120 and the catheter operation member 60 is smaller than the outer diameter of the catheter 12. In addition, the lower support portion 124 is formed in a spring shape in which a flat plate reciprocates in the width direction in a front view. The support structure portion 62D configured in this manner is deformed such that the lower support portion 124 is separated from the base portion 112 (in a direction to be aligned with each other) as the catheter 12 is sandwiched between the base portion 112 and the lower support portion 124 in the assembled state. As a result, the support structure portion 62D can also firmly hold the catheter 12 (with no clearance between the catheter 12 and the support structure portion 62D).

The catheter assembly 10 according to the present embodiment is basically configured as described above, and operations thereof will be described hereinafter.

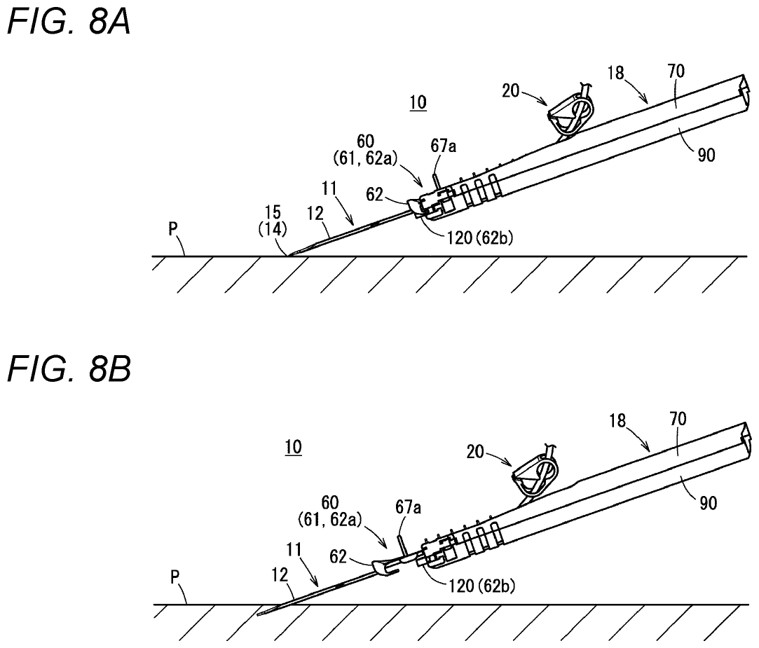

As described above, the catheter assembly 10 is used when infusion, blood transfusion, blood sampling, or the like is performed on the treatment target (living body). As illustrated in FIG. 8A, the user grips and operates the grip 18 to puncture a treatment target P with the multi-structure needle 11 at the time of using the catheter assembly 10.

At the time of puncture, the catheter 12 (multi-structure needle 11) is sandwiched and supported by the catheter operation member 60 and the lower support member 120 at the distal end of the grip 18. At this time, the catheter assembly 10 is in a state in which the catheter 12 (or the support structure portion 62C or 62D itself) is deformed by the support structure portion 62 or 62A to 62D. Therefore, the support structure portion 62 or 62A to 62D can reliably support the catheter 12 on the distal side of the grip 18 at the time of puncture.

Here, a dimensional tolerance occurs when the catheter 12 is manufactured, but the dimensional tolerance at the time of manufacture can be absorbed at a deformed point by adopting the configuration in which the catheter 12 or the support structure portion 62C or 62D itself is deformed. For example, even if the outer diameter of the catheter 12 is manufactured with a minimum tolerance, a wall of the support structure portion 62 or 62A to 62D comes into contact with (or exists at a position sufficiently close to) the catheter 12, and thus, can stably support the outer side of the catheter 12.

Further, even when a force in the upward direction is applied to the catheter operation member 60 from the multi-structure needle 11 in contact with the treatment target P at the time of puncture, the catheter assembly 10 is supported by the support structure portion 62 or 62A to 62D to suppress the deflection of the multi-structure needle 11. As a result, the user can favorably stick the catheter 12 (multi-structure needle 11) into the body.

When the treatment target P is punctured with the multi-structure needle 11 and the needle tip 15 reaches a blood vessel, the user performs an advancing operation of the catheter operation member 60 to advance the catheter 12 beyond the inner needle 14 to be inserted into the blood vessel as illustrated in FIG. 8B. The catheter operation member 60 relatively advances with respect to the lower support member 120 in accordance with the advancing operation of the user. As a result, the catheter 12, the catheter hub 20, and the safety member 40, which are engaged with the catheter operation member 60, also smoothly follow the catheter operation member 60.

The lower support member 120 is rotatable when a proximal end of the side edge 63a of the catheter operation member 60 (operation plate portion 63) comes out of the grip 18. The lower support member 120 relatively rotates with respect to the grip 18 by coming into contact with and being pushed by the hub engagement portion 64 (wall portion 64b) of the catheter operation member 60 in the advancement process. As a result, members (the catheter hub 20 and the safety member 40) on the proximal side of the hub engagement portion 64 can favorably come out of the distal opening portion 18b.

The catheter 12 and the catheter hub 20 are removed from a distal end of the grip 18 by the advancing operation of the user, and then, the safety member 40 protrudes from the distal end of the grip 18. When the catheter operation member 60 is further advanced, the locked protrusion 48 of the safety member 40 moves to the locking portion 100 (an advanced position) of the grip 18. At this time, a distal end of the safety member 40 is exposed from the grip 18 and advances beyond a distal end of the inner needle 14 to cover the inner needle 14, thereby activating an erroneous puncture prevention function.

At the advanced position, the safety member 40 is in a locked state in which movement in both a distal direction and a proximal direction is stopped without coming out of the grip 18. As a result, when the catheter 12, the catheter hub 20, and the catheter operation member 60 further advance, the safety member 40 is separated from these members. The engagement between the catheter operation member 60 and the catheter hub 20 can be released as the catheter operation member 60 and the safety member 40 are separated from each other in the catheter assembly 10. Therefore, the catheter 12 and the catheter hub 20 are separated from the lower side of the catheter operation member 60.

The catheter 12 and the catheter hub 20 are detached from the catheter operation member 60 and indwelled in the treatment target P. After the indwelling, a connector (not illustrated) of another medical device is connected to the catheter hub 20. On the other hand, the inner needle 14, the inner needle hub 30, the safety member 40, and the grip 18 in the state of being integrated are appropriately discarded by the user.

Incidentally, the present invention is not limited to the above-described embodiment, and various modifications can be made in accordance with a gist of the invention. For example, the catheter assembly 10 is not limited to the configuration in which the support structure portion 62 or 62A to 62D is formed by the catheter operation member 60 and the lower support member 120. As an example, the catheter assembly 10 may configure the support structure portion 62 or 62A to 62D between the catheter operation member 60 and the grip 18 (lower grip 90) without including the lower support member 120.

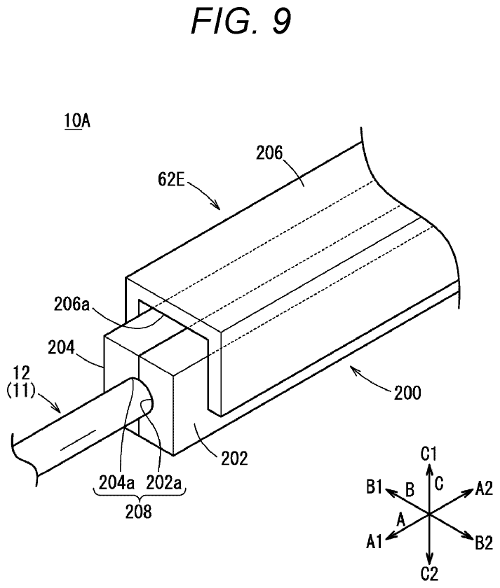

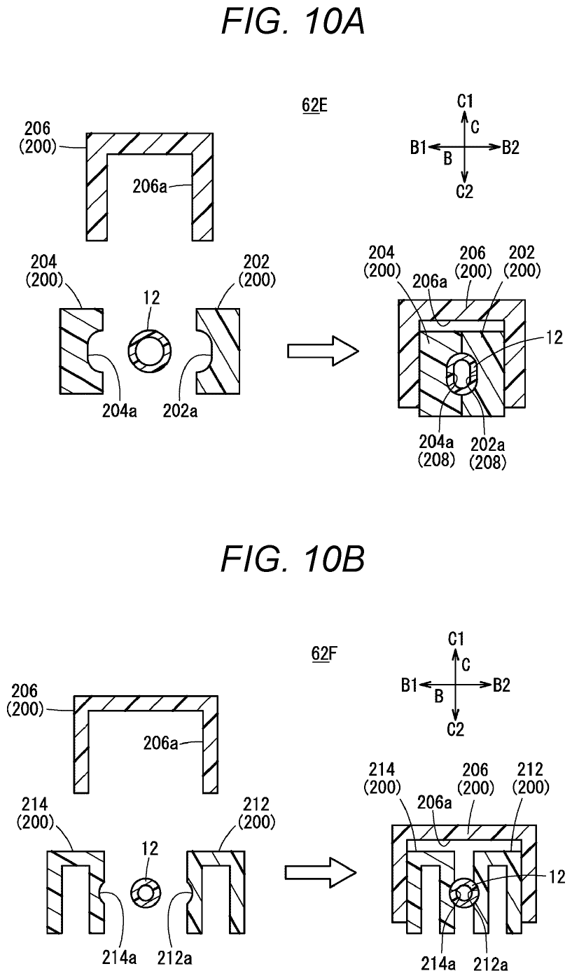

Next, a catheter assembly 10A having a support structure portion 62E or 62F according to a fifth or sixth modification will be described with reference to FIGS. 9, 10A, and 10B. The catheter assembly 10A is different from the above-described catheter assembly 10 in that the catheter 12 is supported at a distal portion of a grip 200 by the support structure portion 62E or 62F provided on the grip 200 itself.

Specifically, a distal end of the grip 200 includes a first site 202 (first support portion 62a) and a second site 204 (second support portion 62b) that can be separated in the width direction (arrow B direction). The first site 202 has a first support groove 202a that supports the catheter 12 on an opposing surface on the arrow B1 side facing the second site 204. On the other hand, the second site 204 has a second support groove 204a that supports the catheter 12 on an opposing surface on the arrow B2 side facing the first site 202.

In addition, the support structure portion 62E includes a sandwiching portion 206 that collectively holds the first and second sites 202 and 204 on the outer side of the first and second sites 202 and 204. The sandwiching portion 206 is formed in a U shape that is open downward, and has a space portion 206a configured to accommodate the first and second sites 202 and 204 on the inner side. The sandwiching portion 206 constitutes, for example, a part of the grip 200 (a portion covering an upper surface of the grip 200), and moves upward or in the distal direction based on an advancing operation of the catheter 12 and the catheter hub 20 to release the sandwiching of the first and second sites 202 and 204.

Alternatively, the sandwiching portion 206 may also be configured using a member different from the grip 200. For example, the sandwiching portion 206 may be configured using the catheter operation member 60, or may be configured using an operation member configured for a user to operate the blunt needle 50 or a guide wire (not illustrated).

In addition, the support structure portion 62E is configured to elastically deform the catheter 12 by assembling the first site 202, the second site 204, and the sandwiching portion 206. That is, in the assembled state in which the first site 202 and the second site 204 are sandwiched by the sandwiching portion 206, the first support groove 202a and the second support groove 204a form a support space 208 having a smaller cross-sectional shape (elliptical shape in the present embodiment) than a perfect circular cross-sectional shape of the catheter 12 before assembly.

As a result, when the catheter 12 is sandwiched in the assembled state, the support structure portion 62E deforms the outer shape of the catheter 12, and the first and second sites 202 and 204 forming the support space 208 are in close contact with the catheter 12 with no clearance. For example, in FIG. 10A, the catheter 12 is deformed into an elliptical shape having a flat portion in the arrow C direction. In this manner, the support structure portion 62E enables the catheter assembly 10A to allow a dimensional tolerance of the catheter 12 and favorably hold the catheter 12.

On the other hand, the support structure portion 62F is different from the support structure portion 62E in that the support structure portion 62F (first and second sites 212 and 214) itself is deformed by assembling the first site 212, the second site 214, and the sandwiching portion 206. Therefore, the first and second sites 212 and 214 are formed in a spring shape elastically deformable in the width direction (arrow B direction) to be aligned with each other, and extend in the width direction before being assembled with the catheter 12 and the sandwiching portion 206.

Further, in a state in which the first and second sites 212 and 214 are sandwiched by the sandwiching portion 206 and the catheter 12 is sandwiched between a first support groove 212a of the first site 212 and a second support groove 214a of the second site 214, each of the first and second sites 212 and 214 contracts to be in close contact with the catheter 12 with no clearance. Therefore, the support structure portion 62F can also allow a dimensional tolerance of the catheter 12 and favorably support the catheter 12. Note that both the first site 212 and the second site 214 of the support structure portion 62F are not limited to the configuration in which the both are deformed but may adopt a configuration in which either one is deformed.

In addition, the configuration example in which any one of the catheter 12 and the support structure portion 62 or 62A to 62F is deformed has been described as above, but it is a matter of course that the catheter assembly 10 or 10A may have a configuration in which both the catheter 12 and the support structure portion 62 or 62A to 62F are deformed.

Technical ideas and effects that can be grasped from the above-described embodiment are described as follows.

The catheter assembly 10 or 10A according to an aspect of the present invention includes: the catheter 12; the catheter hub 20 fixed to the catheter 12; the inner needle 14 inserted through the catheter 12; the grip 18 that fixes and holds the inner needle 14; and the support structure portion 62 or 62A to 62F that holds the outer side of the catheter 12 in an assembled state. In the assembled state, the support structure portion 62 or 62A to 62F sandwiches the catheter 12, and at least one of the support structure portion 62 or 62A to 62F and the catheter 12 is elastically deformed.

The above-described catheter assembly 10 or 10A can favorably support the outer side of the catheter 12 since at least one of the support structure portion 62 or 62A to 62F itself and the catheter 12 is elastically deformed due to the sandwiching of the catheter 12. That is, even if the catheter 12 is formed to be thin within the range of the tolerance, the tolerance can be absorbed in a deformed portion of the support structure portion 62 or 62A to 62F or the catheter 12. Therefore, the catheter assembly 10 or 10A can suppress the deflection of the catheter 12 by the support structure portion 62 or 62A to 62F at the time of puncture, and can enhance the operability of the user.

In addition, the support structure portion 62 or 62A to 62F include the first support portion 62a that supports the outer side of the catheter 12 and the second support portion 62b that has at least a part being located at a position facing the first support portion 62a and supports the outer side of the catheter 12. As a result, the catheter assembly 10 or 10A can easily form a supporting state in which the catheter 12 is sandwiched between the first support portion 62a and the second support portion 62b.

In addition, at least one of the first support portion 62a and the second support portion 62b is deformed in a direction to be aligned with each other in the assembled state. As a result, the catheter assembly 10 or 10A can stably deform the support structure portion 62D or 62F in the supporting state in which the catheter 12 is sandwiched between the first support portion 62a and the second support portion 62b, and can smoothly release the supporting state when the catheter 12 advances or the like.

In addition, the support structure portion 62C, 62D, or 62F includes elastic portions (the pair of ridge portions 113, the lower support portion 124, the first site 212, and the second site 214) that are elastically deformed in a contact state with the catheter 12. As a result, the support structure portion 62C, 62D, or 62F can be more favorably deformed with respect to the catheter 12.

In addition, the catheter 12 is deformed to have a flat portion in at least one direction by the support structure portion 62, 62A, 62B, or 62E. As a result, the support structure portion 62, 62A, 62B, or 62E can easily deform the catheter 12 by sandwiching the catheter 12 from the one direction.

In addition, the catheter 12 is deformed to have a flat portion in another direction orthogonal to the one direction by the support structure portion 62. As a result, the support structure portion 62 can greatly deform the catheter 12 in the one direction and the another direction and more firmly support the catheter 12.

In addition, the support structure portion 62 has the base portion 112 in contact with the outer peripheral surface of the catheter 12 and a pair of projecting portions (distal projecting portions 113a) protruding from the base portion 112 and causing the catheter 12 to be located therebetween. The catheter assembly 10 can prevent the catheter 12 from coming out in a direction in which the pair of projecting portions is arranged by locating the catheter 12 between the pair of projecting portions.

In addition, the pair of projecting portions (distal projecting portions 113a) is located on the distal side of the grip 18. As a result, the catheter assembly 10 can smoothly move a member having the pair of projecting portions in the distal direction with respect to the grip 18 while preventing the catheter 12 from coming out.

In addition, the support structure portion 62 or 62A to 62D includes the operation unit 61 configured to operate the relative movement of the catheter 12 with respect to the grip 18. As a result, the catheter assembly 10 can smoothly advance the catheter 12 with the advancement of the operation unit 61 while continuing the support of the catheter 12 by the support structure portion 62 or 62A to 62D.

In addition, the support structure portion 62E or 62F includes the sandwiching portion 206 that holds a plurality of sites, configured to support the catheter 12 and be separable, in a sandwiched state. Since the sandwiching portion 206 is provided in this manner, the support structure portion 62E or 62F can more stably maintain the supporting state of the catheter 12.

In addition, the sandwiching portion 206 is a part of the grip 200. In this manner, the catheter assembly 10A can reliably maintain the supporting state of the catheter 12 by the grip 200.

What is claimed is:

1. A catheter assembly comprising:
a catheter;
a catheter hub fixed to the catheter;
an inner needle inserted through the catheter;
a grip that fixes and holds the inner needle; and
a support structure portion that holds an outer side of the catheter in an assembled state;
wherein, in the assembled state, the support structure portion sandwiches the catheter, and at least one of the support structure portion or the catheter is elastically deformed.

2. The catheter assembly according to claim 1, wherein:
the support structure portion comprises:
a first support portion that supports the outer side of the catheter, and
a second support portion, at least a part of which is located at a position facing the first support portion and supports the outer side of the catheter.

3. The catheter assembly according to claim 2, wherein:
at least one of the first support portion or the second support portion is deformed in a direction in which the first support portion and the second support portion are aligned with each other in the assembled state.

4. The catheter assembly according to claim 1, wherein:
the support structure portion includes an elastic portion that is elastically deformed in a contact state with the catheter.

5. The catheter assembly according to claim 1, wherein:
the catheter is deformed by the support structure portion to have a first flat portion extending in a first direction.

6. The catheter assembly according to claim 5, wherein
the catheter is deformed by the support structure portion to have a second flat portion extending in a second direction orthogonal to the first direction.

7. The catheter assembly according to claim 1, wherein:
the support structure portion comprises:
a base portion in contact with an outer peripheral surface of the catheter, and
a pair of projecting portions protruding from the base portion between which the catheter is located.

8. The catheter assembly according to claim 7, wherein:
the pair of projecting portions is located on a distal side of the grip.

9. The catheter assembly according to claim 1, wherein:
the support structure portion comprises an operation unit configured to operate relative movement of the catheter with respect to the grip.

10. The catheter assembly according to claim 1, wherein:
the support structure portion comprises:
a plurality of support portions that are separable from one another and that support the outer side of the catheter, and
a sandwiching portion that holds the plurality of support portions in a sandwiched state.

11. The catheter assembly according to claim 10, wherein:
the sandwiching portion is a part of the grip.

12. A catheter assembly comprising:
a catheter;
a catheter hub fixed to the catheter;
an inner needle inserted through the catheter;
a grip that fixes and holds the inner needle; and
a support structure portion that holds an outer side of the catheter in an assembled state;
wherein, in the assembled state, the support structure portion sandwiches the catheter and is elastically deformed by the catheter.

13. The catheter assembly according to claim 12, wherein:
the support structure portion comprises:
a first support portion that supports the outer side of the catheter, and
a second support portion, at least a part of which is located at a position facing the first support portion and supports the outer side of the catheter.

14. The catheter assembly according to claim 13, wherein:
at least one of the first support portion or the second support portion is deformed in a direction in which the first support portion and the second support portion are aligned with each other in the assembled state.

15. The catheter assembly according to claim 12, wherein:
the support structure portion includes an elastic portion that is elastically deformed in a contact state with the catheter.

16. A catheter assembly comprising:
a catheter;
a catheter hub fixed to the catheter;
an inner needle inserted through the catheter;
a grip that fixes and holds the inner needle; and
a support structure portion that holds an outer side of the catheter in an assembled state;
wherein, in the assembled state, the support structure portion sandwiches the catheter, and the catheter is elastically deformed.

17. The catheter assembly according to claim 16, wherein:
the catheter is deformed by the support structure portion to have a first flat portion extending in a first direction.

18. The catheter assembly according to claim 17, wherein
the catheter is deformed by the support structure portion to have a second flat portion extending in a second direction orthogonal to the first direction.

19. The catheter assembly according to claim 16, wherein:
the support structure portion comprises:
a base portion in contact with an outer peripheral surface of the catheter, and
a pair of projecting portions protruding from the base portion between which the catheter is located.

20. The catheter assembly according to claim 19, wherein:
the pair of projecting portions is located on a distal side of the grip.
